(19) United States
(12) Patent Application Publication
(10) Pub. No.: US 2018/0319800 A1
(43) Pub. Date: Nov. 8, 2018

(54) (1R,4R) 7-OXO-2-AZABICYCLO[2.2.2]OCT-5-ENE AND DERIVATIVES THEREOF

(71) Applicant: DemeRx, Inc., Miami, FL (US)

(72) Inventor: Robert M. Moriarty, Michiana Shores, IN (US)

(73) Assignee: DemeRx, Inc., Miami, FL (US)

(21) Appl. No.: 15/811,549

(22) Filed: Nov. 13, 2017

Related U.S. Application Data
(63) Continuation of application No. 13/749,592, filed on Jan. 24, 2013, now abandoned.
(60) Provisional application No. 61/741,798, filed on Jan. 25, 2012.

(51) Int. Cl. C07D 471/08; C07D 491/20; C07D 453/06; C07D 495/20; C07D 493/20; A61K 31/46

(52) U.S. Cl. CPC C07D 471/08; C07D 491/20; C07D 453/06; A61K 31/46; C07D 495/20; C07D 493/20

(57) ABSTRACT

This invention provides novel (1R,4R) 7-oxo-2-azabicyclo[2.2.2]oct-5-ene and derivatives thereof, preferably in substantially enantiomerically enriched forms, intermediates thereto, and processes of their synthesis.

### CROSS REFERENCE TO RELATED APPLICATION

This application claims the benefit under 35 U.S.C. 119(e) of U.S. Provisional Application Ser. No. 61/741,798 filed Jan. 25, 2012, which is hereby incorporated by reference into this application in its entirety.

### FIELD OF THE INVENTION

This invention provides (1R,4R) 7-oxo-2-azabicyclo[2.2.2]oct-5-ene as well as derivatives thereof. Such compounds are readily converted into pharmaceutically important compounds containing the isoquinuclidene moiety. In one embodiment, the 7-oxo-2-azabicyclo[2.2.2]oct-5-ene compounds of this invention are in substantially enantiomerically enriched forms. This invention also provides for processes for preparing such 7-oxo-2-azabicyclo[2.2.2]oct-5-ene compounds as well as for preparing novel intermediates used therein.

### BACKGROUND OF THE INVENTION

Many pharmaceutical compounds mirror the structures of natural products. In particular, certain aspects of the natural product are modified in order to enhance beneficial properties and/or to minimize detrimental properties. The portion of the natural product which imparts some or all of the pharmaceutical activity is referred to as a “pharmacophore”. One example of a potent pharmacophore found in nature is the structurally complex chiral isoquinuclidene moiety which has a core structure:

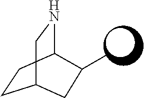

where [symbol]
denotes a non-hydrogen substituent. This structure is common in pharmacologically active natural products, such as the Iboga alkaloids.

Synthesizing compounds to include the isoquinuclidene moiety, especially in a substantially enantiomerically pure form is a challenging task. Heretofore, Iboga alkaloids, such as ibogaine, were conventionally prepared from one of its naturally occurring precursors such as voacangine. In turn, voacangine is obtained from plants, whose supply is limited and where the quality of the supply is unpredictable.

Synthesizing non-natural compounds including the structurally complex isoquinuclidene moiety, such as those used as pharmaceutically active agents, is also challenging. For non-natural isoquinuclidenes as 5-HT3 ligands, see, Iriepa et al., Bioorg. Med. Chem. Lett. 12, 2002, 189-192. See also Glick, et al., U.S. Pat. No. 6,211,360 which discloses a variety of complex compounds having a carboxyl substituted isoquinuclidene ring or a derivative of that carboxyl substitution.

### SUMMARY OF THE INVENTION

Provided herein is a novel 7-oxo-2-azabicyclo[2.2.2]oct-5-ene having 1R,4R stereochemistry and derivatives thereof, which can be converted into substantially more complex compounds having the isoquinuclidene moiety. In one embodiment, these compounds (as well as their intermediates) are provided in substantially enantiomerically pure forms so as to provide for entry into various pharmacologically active products, containing an isoquinuclidene moiety as found for example in 5-HT3 ligands (see, Iriepa et al., supra).

Also provided herein are processes for preparing the 7-oxo-2-azabicyclo[2.2.2]oct-5-ene derivatives, and intermediates thereto, preferably in substantially enantiomerically enriched forms.

### BRIEF DESCRIPTION OF THE FIGURES

FIG. 1 illustrates a ¹H-NMR spectrum in CDCl₃ of compound 10,

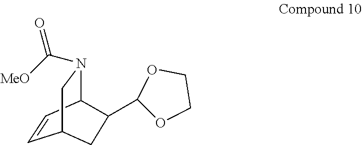 [SMILES: COC(=O)N1CC2C=CC1C(C1OCCO1)C2]

which is an N-protected, 5 membered cyclic ketal of R,R 7-oxo-2-azabicyclo[2.2.2]oct-5-ene.

FIG. 2 illustrates a ¹H-NMR spectrum in CDCl₃ of compound 11,

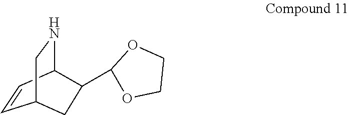 [SMILES: C1=CC2NCC1CC2C1OCCO1]

which is a 5 membered cyclic ketal of R,R 7-oxo-2-azabicyclo[2.2.2]oct-5-ene.

### DETAILED DESCRIPTION OF THE INVENTION

This invention relates to 1R,4R 7-oxo-2-azabicyclo[2.2.2]oct-5-ene and derivatives thereof as well as to processes for preparing them. Before this invention is described in greater detail, the following terms will be defined.

As used herein and in the appended claims, the singular forms “a”, “an”, and “the” include plural referents unless the context clearly dictates otherwise. Thus, for example, reference to “a salt” includes a plurality of such salts.

### Definitions

As used herein, “alkenyl” refers to hydrocarbyl groups having from 2 to 10 carbon atoms and at least one and up to 3 carbon carbon double bonds. Examples of alkenyl include vinyl, allyl, dimethyl allyl, and the like.

As used herein, “alkoxy” refers to —O-alkyl.

As used herein, “alkyl” refers to hydrocarbyl groups having from 1 to 10 carbon atoms, more preferably 1 to 6 carbon atoms, and still more preferably 1-4 carbon atoms. The alkyl group may contain linear or branched carbon chains. This term is exemplified by groups such as methyl, ethyl, n-propyl, iso-propyl, n-butyl, t-butyl, n-pentyl, n-decyl and the like.

As used herein, “alkynyl” refers to hydrocarbyl groups having from 2 to 10 carbon atoms and at least one and up to 2 carbon carbon triple bonds. Examples of alkynyl include ethynyl, propargyl, dimethylpropargyl, and the like.

As used herein, “amino” refers to —NRₓR_{y} wherein each R^{x} and R^{y} independently is hydrogen, C₁-C₆ alkyl, C₂-C₆ alkenyl, C₂-C₆ alkynyl, C₆-C₁₀ aryl, C₃-C₈ cycloalkyl, C₂-C₁₀ heteroaryl, and C₃-C₈ heterocyclyl.

As used herein, “aryl” refers to an aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom.

As used herein, “Cₓ” refers to a group having x carbon atoms, wherein x is an integer, for example, C₄ alkyl refers to an alkyl group having 4 carbon atoms.

As used herein, “cycloalkyl” refers to cyclic hydrocarbyl groups of from 3 to 10 carbon atoms having single or multiple condensed rings, which condensed rings may be aromatic or contain a heteroatom, provided that the point of attachment is at a cycloalkyl carbon atom. Cycloalkyl includes, by way of example, adamantyl, cyclopropyl, cyclobutyl, cyclopentyl, cyclooctyl and the like. Cycloalkyl rings are preferably saturated, though, cycloalkyl rings including 1-2 carbon carbon double bonds are also contemplated provided that the ring is not aromatic.

As used herein, “chiral Lewis acid” refers to a Lewis acid, which is complexed with, such as, for example, covalently bound with, a chiral compound that can bind to the Lewis acid. Such Lewis acids include halide and alkoxides of titanium (IV), and such other metals. Suitable chiral compounds include various diols and amino alcohols, such as binol, taddol, and the like, and are well known in the art.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, “ee” refers to enantiomeric excess and is expressed as (e¹-e²)% where e¹ and e² are the two enantiomers. For example, if the % of e¹ is 95 and the % of e² is 5, then the e¹ enantiomer is present in an ee of 90%. The ee of an enantiomer in a mixture of enantiomers is determined following various methods well known to the skilled artisan, such as using chiral lanthanide based nuclear magnetic resonance shift reagents, forming derivatives with chiral compounds such as chiral hydroxyacids, amino acids, and the like. Various physical measurements such as circular dichroism, optical rotation, etc. are also useful in determining the ee of a mixture of enantiomers.

As used herein, —CO₂H “ester” refers to —CO₂RE wherein RE is selected from the group consisting of C₆-C₁₀ aryl and C₁-C₆ alkyl optionally substituted with 1-3 C₆-C₁₀ aryl groups.

As used herein, “halo” refers to F, Cl, Br, or I.

As used herein, “heteroaryl” refers to an aromatic group of from 1 to 10 carbon atoms and 1 to 4 heteroatoms selected from the group consisting of oxygen, nitrogen, sulfur within the ring, wherein the nitrogen and/or sulfur atom(s) of the heteroaryl are optionally oxidized (e.g., N-oxide, —S(O)— or —S(O)₂—), provided that the ring has at least 5 ring atoms and up to 14, or preferably from 5-10, ring atoms. Such heteroaryl groups can have a single ring (e.g., pyridyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. Examples of heteroaryls include pyridyl, pyrrolyl, indolyl, thiophenyl, furyl, and the like.

As used herein, “heterocyclyl” or heterocycle refers to a cycloalkyl group of from 1 to 10 carbon atoms and 1 to 4 heteroatoms selected from the group consisting of oxygen, nitrogen, sulfur within the ring, wherein the nitrogen and/or sulfur atom(s) of the heteroaryl are optionally oxidized (e.g., N-oxide, —S(O)— or —S(O)₂—), provided that the ring has at least 3 and up to 14, or preferably from 5-10 ring atoms. Such heterocyclyl groups can have a single ring or multiple condensed rings wherein the condensed rings may not contain a heteroatom and/or may contain an aryl or a heteroaryl moiety, provided that the point of attachment is through an atom of the non-aromatic heterocyclyl group. Examples of heterocyclyl include pyrrolidinyl, piperadinyl, piperazinyl, and the like. Heterocyclyl rings are preferably saturated, though, heterocyclyl rings including 1-2 carbon carbon double bonds are also contemplated provided that the ring is not aromatic.

As used herein, “olefin metathesis reagent” refers to well known reagents that are employed, preferably in catalytic amounts, for ring closing olefin metathesis, as schematically shown below

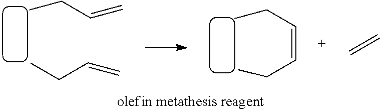

Exemplary olefin metathesis reagents include, without limitation, various commercially available, for example from Sigma-Aldrich, Grubbs' catalysts, such as:

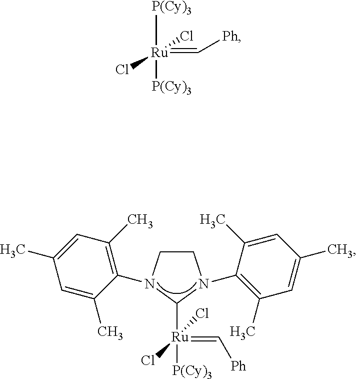

or their immobilized version, such as:

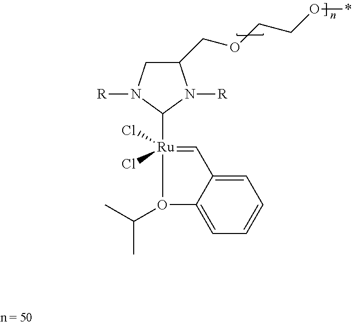

In certain embodiments, commercially available (for example from Strem Chemicals, Inc.) molybdenum based Schrock's catalysts, such as:

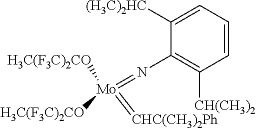

are also useful as olefin metathesis reagent.

As used herein, “protecting group” or “Pg” refers to well known functional groups which, when bound to a functional group, render the resulting protected functional group inert to the reaction to be conducted on other portions of the compound and the corresponding reaction condition, and which can be reacted to regenerate the original functionality under deprotection conditions. The protecting group is selected to be compatible with the remainder of the molecule. In one embodiment, the protecting group is an “amine protecting group” which protects an —NH— or an —NH₂— moiety, for example during the syntheses described here. Examples of amine protecting groups include, for instance, benzyl, acetyl, oxyacetyl, carbonyloxybenzyl (Cbz), Fmoc, and the like. In another embodiment, the protecting group is a “hydroxy protecting group” which protects a hydroxyl functionality during the synthesis described here. Examples of hydroxyl protecting groups include, for instance, benzyl, p-methoxybenzyl, p-nitrobenzyl, allyl, trityl, dialkylsilylethers, such as dimethylsilyl ether, and trialkylsilyl ethers such as trimethylsilyl ether, triethylsilyl ether, and t-butyldimethylsilyl ether; esters such as benzoyl, acetyl, phenylacetyl, formyl, mono-, di-, and trihaloacetyl such as chloroacetyl, dichloroacetyl, trichloroacetyl, trifluoroacetyl; and carbonates such as methyl, ethyl, 2,2,2-trichloroethyl, allyl, and benzyl. Examples of keto protecting groups include linear and cyclic ketals and Schiff's bases. As the skilled artisan would appreciate, one or more of these protecting groups are also useful as amine protecting groups. Additional examples of amine, hydroxy, and keto protecting groups are found in standard reference works such as Greene and Wuts, Protective Groups in Organic Synthesis, 2d Ed., 1991, John Wiley & Sons, and McOmie Protective Groups in Organic Chemistry, 1975, Plenum Press. Methods for protecting and deprotecting hydroxyl, —NH—, —NH₂—, and keto groups disclosed herein can be found in the art, and specifically in Greene and Wuts, supra, and the references cited therein.

As used herein, “silyl” refers to Si(R^{z})₃ wherein each R^{Z} independently is C₁-C₆ alkyl or C₆-C₁₀ aryl.

As used herein, “substantially enantiomerically enriched,” “substantially enantiomerically pure” and grammatical equivalents thereof refers to an enantiomer in an enantiomeric mixture with at least 95% ee, preferably 98% ee, or more preferably 99% ee.

### Compounds of the Invention

In one aspect, this invention provides a compound of Formula (I) or (Ia):

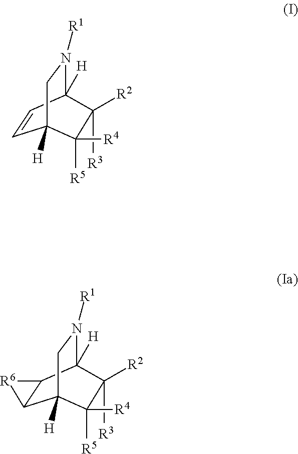

or a salt thereof wherein,

R¹ is selected from the group consisting of hydrogen, —CO₂R¹¹, —COR¹², —C(R¹³)₃, and another amine protecting group;

R¹¹ is selected from the group consisting of C₁-C₆ alkyl optionally substituted with 1-3 substituents selected from the group consisting of C₆-C₁₀ aryl, C₃-C₈ cycloalkyl, C₂-C₁₀ heteroaryl, C₃-C₈ heterocyclyl, halo, amino, —N₃, hydroxy, C₁-C₆ alkoxy, silyl, nitro, cyano, and CO₂H or an ester thereof, C₂-C₆ alkenyl, C₂-C₆ alkynyl, C₆-C₁₀ aryl, C₂-C₁₀ heteroaryl, C₃-C₈ cycloalkyl, and C₃-C₈ heterocyclyl;

R¹² and R¹³ independently are selected from the group consisting of hydrogen, C₁-C₆ alkyl optionally substituted with 1-3 substituents selected from the group consisting of C₆-C₁₀ aryl, C₃-C₈ cycloalkyl, C₂-C₁₀ heteroaryl, C₃-C₈ heterocyclyl, halo, amino, —N₃, hydroxy, C₁-C₆ alkoxy, silyl, nitro, cyano, and CO₂H or an ester thereof, C₂-C₆ alkenyl, C₂-C₆ alkynyl, C₆-C₁₀ aryl, C₂-C₁₀ heteroaryl, C₃-C₈ cycloalkyl, and C₃-C₈ heterocyclyl;

R² and R³ independently are selected from the group consisting of hydrogen, hydroxy, C₁-C₆ alkyl, C₂-C₆ alkenyl, and C₂-C₆ alkynyl, —SR²¹ and —OR²², wherein the alkyl, alkenyl, or the alkynyl group is optionally substituted with 1-3 substituents selected from the group consisting of keto, halo, C₁-C₆ alkoxy, amino, hydroxy, cyano, nitro, —NHCOCH₃, —N₃, and —CO₂H or an ester thereof, provided that at least one of R² and R³, preferably R² is a non-hydrogen substituent, or

R² and R³ together with the carbon atom to which they are bonded to form a keto (C═O) group, a Schiff base (═NR²⁴), a vinylidene moiety of formula ═CR²⁵R²⁶, or form a 5-6 membered cyclic ketal or thioketal, which cyclic ketal or thioketal is of formula:

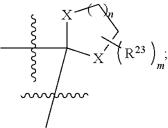

each R²¹ is independently selected from the group consisting of C₁-C₆ alkyl optionally substituted with 1-3 substituents selected from the group consisting of C₆-C₁₀ aryl, C₃-C₈ cycloalkyl, C₂-C₁₀ heteroaryl, C₃-C₈ heterocyclyl, halo, amino, —N₃, hydroxy, C₁-C₆ alkoxy, silyl, nitro, cyano, and CO₂H or an ester thereof, C₂-C₆ alkenyl, C₂-C₆ alkynyl, C₆-C₁₀ aryl, C₂-C₁₀ heteroaryl, C₃-C₈ cycloalkyl, and C₃-C₈ heterocyclyl;

each R²² is independently selected from the group consisting of C₁-C₆ alkyl optionally substituted with 1-3 substituents selected from the group consisting of C₆-C₁₀ aryl, C₃-C₈ cycloalkyl, C₂-C₁₀ heteroaryl, C₃-C₈ heterocyclyl, halo, amino, —N₃, hydroxy, C₁-C₆ alkoxy, silyl, nitro, cyano, and CO₂H or an ester thereof, C₂-C₆ alkenyl, and C₂-C₆ alkynyl;

where X in both occurrences is either oxygen or sulfur;

m is 1, 2, 3, or 4;

n is 1 or 2;

R²³ is selected from the group consisting of C₁-C₆ alkyl and C₆-C₁₀ aryl;

R²⁴ is selected from the group consisting of C₆-C₁₀ aryl and C₂-C₁₀ heteroaryl;

R²⁵ is hydrogen, C₁-C₆ alkyl, C₂-C₆ alkenyl, and C₂-C₆ alkynyl, wherein the alkyl, alkenyl, or the alkynyl group is optionally substituted with 1-3 substituents selected from the group consisting of keto, C₁-C₆ alkoxy, amino, hydroxy, cyano, nitro, —NHCOCH₃, and —CO₂H or an ester thereof;

R²⁶ is hydrogen or C₁-C₆ alkyl;

R⁴ and R⁵ independently are selected from the group consisting of hydrogen, halo, and C₁-C₆ alkyl optionally substituted with 1-3 substituents selected from C₆-C₁₀ aryl, C₃-C₈ cycloalkyl, C₂-C₁₀ heteroaryl, C₃-C₈ heterocyclyl, halo, amino, —N₃, hydroxy, C₁-C₆ alkoxy, silyl, nitro, cyano, vinyl, ethynyl, and CO₂H or an ester thereof,

R⁶ is selected from the group consisting of —O—, —NH—, and —NR⁶¹;

R⁶¹ is selected from the group consisting of hydrogen, —SO₂R⁶², and an amine protecting group;

R⁶² is selected from the group consisting of C₁-C₆ alkyl optionally substituted with 2-5 halo groups and C6-C10 aryl optionally substituted with 1-3 C₁-C₆ alkyl and halo groups;

the amine protecting group is selected from the group consisting of —CO₂CMe₃, —CO₂Bn, —CO₂-allyl, -Fmoc (flurenyloxymethyl), —COCF₃, Bn (CH₂Ph), —CHPh₂, and —CPh₃; and wherein the cycloalkyl, heterocyclyl, aryl, or heteroaryl, is optionally substituted with 1-3 substituents selected from the group consisting of C₁-C₆ alkyl, C₂-C₆ alkenyl, C₂-C₆ alkynyl, C₆-C₁₀ aryl, C₃-C₈ cycloalkyl, C₂-C₁₀ heteroaryl, C₃-C₈ heterocyclyl, halo, amino, —N₃, hydroxy, C₁-C₆ alkoxy, silyl, nitro, cyano, and CO₂H or an ester thereof.

As used herein, a salt refers to preferably a salt of a mineral acid, or an organic acid such as a carboxylic acid or a sulfonic acid, and/or to alkali, alkaline earth, and various ammonium (including tetraalkyl ammonium, pyridinum, imidazolium and the like) salts. Non limiting examples of acid salts include salts of hydrochloric acid, hydrobromic acid, phosphoric acid, sulfuric acid, methane sulfonic acid, phosphorous acid, nitric acid, perchloric acid, acetic acid, tartaric acid, lactic acid, succinic acid, and citric acid.

As used herein, compounds of this invention include tautomers thereof, including without limitation, keto enol, —NH—CO—N═COH—, and such other tautomers.

In another embodiment, the compound is of Formula (II):

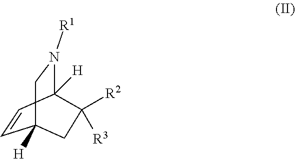

wherein R¹, R², and R³ are defined as in Formula (I) above.

For the compound of Formula (II), in a preferred embodiment, CR²R³ is a protected ketone, more preferably, a cyclic ketal or thioketal. Within these embodiments, in a preferred embodiment, R¹ is hydrogen.

In another embodiment, the compound is of formula (II):

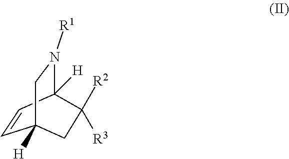

wherein R¹ is —CO₂R¹¹, —COR¹², —C(R³)₃, or another amine protecting group. In another embodiment, R¹¹ and R¹² are independently methyl, ethyl, propyl, isopropyl, butyl, isobutyl, or tertiary butyl. In another embodiment, R² is C₁-C₆ alkyl, C₂-C₆ alkenyl, or C₂-C₆ alkynyl, wherein the alkyl, alkenyl, or the alkynyl group is optionally substituted with 1-3 substituents selected from the group consisting of keto, halo, C₁-C₆ alkoxy, amino, hydroxy, cyano, nitro, —NHCOCH₃, —N₃, and —CO₂H or an ester thereof. In another embodiment, R³ is hydroxy. In another embodiment, R³ is hydrogen.

In another embodiment, the compound is of Formula (IIA):

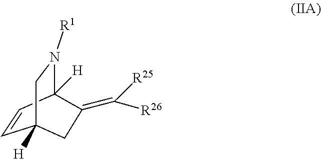

wherein R¹, R²⁵, and R²⁶ are defined as in Formula (I) above. In another embodiment, R¹ is —CO₂R¹¹, —COR¹², —C(R¹³)₃, and another amine protecting group. In another embodiment, R¹¹ and R¹² are independently methyl, ethyl, propyl, isopropyl, butyl, isobutyl, or tertiary butyl. In another embodiment, R²⁵ is C₁-C₆ alkyl, C₂-C₆ alkenyl, and C₂-C₆ alkynyl, wherein the alkyl, alkenyl, or the alkynyl group is optionally substituted with 1-3 substituents selected from the group consisting of keto, C₁-C₆ alkoxy, amino, hydroxy, cyano, nitro, —NHCOCH₃, and —CO₂H or an ester thereof. In one embodiment, R²⁶ is hydrogen.

In another embodiment, the compound is of Formula (III):

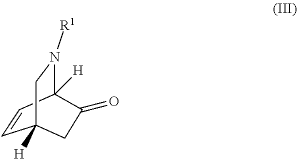

wherein R¹ is defined as in Formula (I) above, and is preferably a non-hydrogen substituent. In another embodiment, for the compound of Formula (III), R¹ is CO₂R¹¹ or another amine protecting group as defined herein, and R¹¹ is C₁-C₆ alkyl.

In another embodiment, this invention provides compounds of the formula:

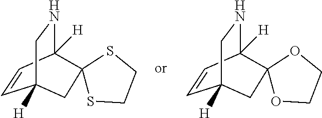 [SMILES: C1=C[C@@H]2CNC1C1(C2)SCCS1 ; C1=C[C@@H]2CNC1C1(C2)OCCO1]

or a salt thereof. In another embodiment, the compound is an R,R enantiomer. In another embodiment, the compound is in substantial enantiomeric excess (ee).

### Processes of the Invention

The compounds of this invention are prepared following novel processes provided herein and obvious modifications of synthetic methods well known to the skilled artisan upon appropriate substitution of starting material and reagents, and/or following methods that will become apparent to the skilled artisan upon reading this disclosure.

Accordingly, the compounds of this invention can be prepared from readily available starting materials using the general processes and procedures described and illustrated herein. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis. Third Edition. Wiley, New York, 1999, and references cited therein.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989). In one of its process aspects, this invention provides a process for preparing a compound of Formula (II)

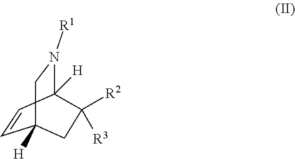

or a salt thereof, wherein R¹, R², and R³ are defined as in Formula (I) or in any aspect or embodiment here, which process comprises contacting a compound of Formula (IV):

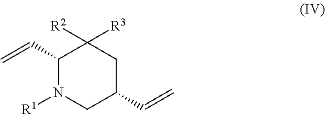

or a salt thereof with from 0.1-10 molar equivalent, preferably less than 1 molar equivalent of an olefin metathesis reagent under conditions to provide the compound of Formula (II) or a salt thereof.

Such conditions include the use of a suitable inert solvent, such as for example chlorinated solvent such as dichloromethane, a temperature of from 15° C. to 40° C., and reaction times of from 0.5 h to 1 day. Preferably, the reaction is carried out for a period of time sufficient to provide a substantial amount of the product, which can be ascertained by using routine methods such as thin layer chromatography, ¹H-nuclear magnetic resonance (NMR) spectroscopy, and the likes. The products can be isolated and optionally purified using standard purification techniques, such as liquid chromatography, crystallization, precipitation, and distillation under reduced pressure, or the products may be used for a subsequent reaction without further purification.

The synthesis of the compounds of this invention following the processes of this invention are schematically shown below.

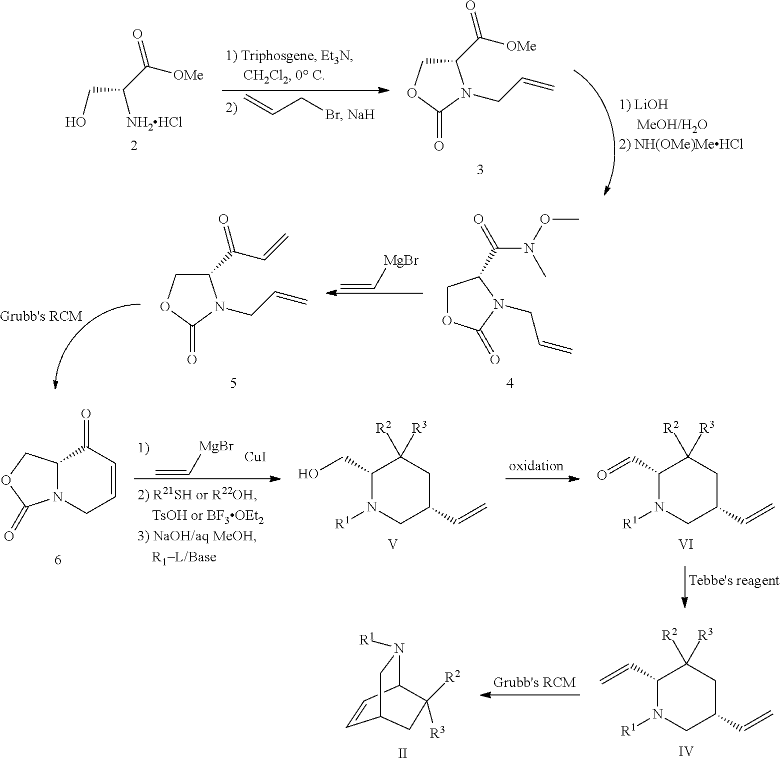

The first step of the process uses, as the chiral element, D-serine methyl ester (2), which is reacted with triphosgene or another phosgene source, in the presence of a base, and further with an allylating agent and another base, preferably a hydride, to provide (R)-2-oxo oxazolidine-4-carboxylic acid methyl ester). Preferably the reactions are carried out in a solvent that is inert to the reactant and reagents. The use of an immobilized, resin-bound via the carboxyl moiety-serine ester is also contemplated as the starting material to reduce potential product loss during aqueous work up. The N-allylation, introduces one of the requisite alkenes (3) to the molecule.

The second alkene results from the Weinreb amide procedure to yield the vinyl ketone (5). Accordingly, compound 3 is hydrolyzed using aqueous alkali and converted to its N-methoxy amide (4). Compound 4 is reacted with a vinyl anion equivalent, such as vinyl magnesium bromide, in a solvent such as ether or tetrahydrofuran, preferably at a temperature of −5-10° C. to provide compound 5.

The first Grubbs reaction on 5 affords the chiral oxazolidinone (6). Conjugate addition of vinyl magnesium bromide in presence of a copper (I) salt such as CuI, protection of the keto group, alkaline oxazolidine ring cleavage, alkylation or acylation with R¹-L, where L is a leaving group, such as e.g. a halo or a mesylate, tosylate, or such other group, provides compound V. Compound V is selectively oxidized to an aldehyde to provide compound VI. Various art known oxidative methods including pyridinium chlorochromate, Swern oxidation, N-methyl morphomine —N-oxide (NMO) and perruthenate, are useful for the selective oxidation. Olefination of compound VI using Tebbe's reagent or a Wittig reaction yields the 1,5 divinyl substituted piperidine (IV). Grubbs cyclization of compound IV yields compound II. When R¹ is hydrogen, and CR²R³ is:

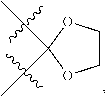 [SMILES: *C1(*)OCCO1]

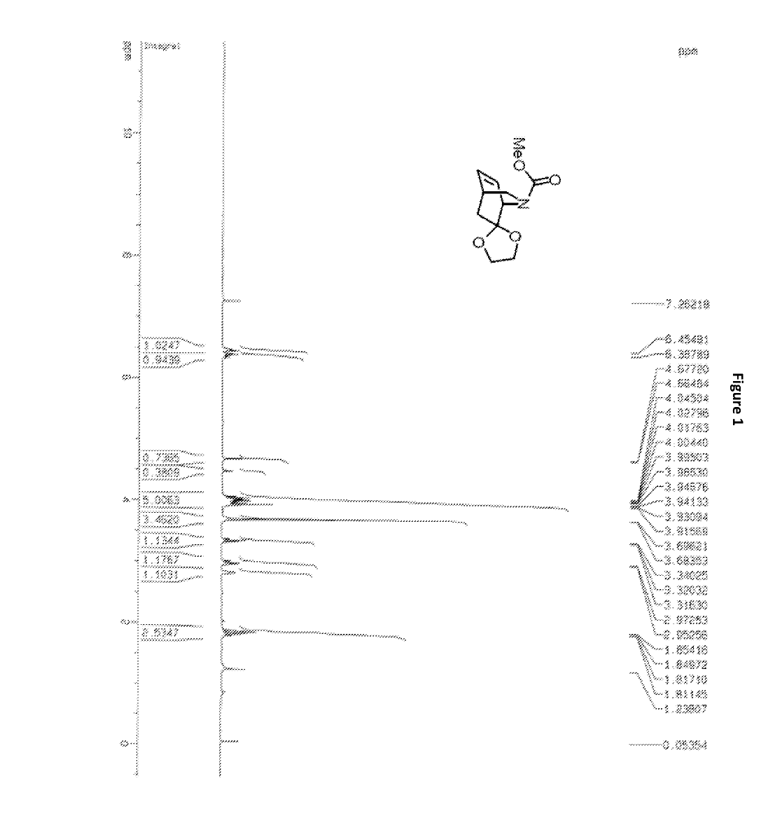

the ¹H-NMR of the resulting compound, compound 10, is shown in FIG. 1.

Compounds of Formulas (IIIA) and (IIB) are synthesized from a compound of formula (II) wherein CR²R³ is keto following a reaction, e.g., with an alkyl anion (R²(−)) or with a Wittig reagent (Ph₃P═CR²⁵SR²⁶), as are well known to the skilled artisan. The compound wherein R³ is OH is converted to one wherein R³ is hydrogen by well known reaction such as by dehydration-hydrogenation. As to the compounds where CR²R³ is C═CR²⁵R²⁶, they can be hydrogenated employing catalytic hydrogenation procedures well known to the skilled artisan such that the hydrogenation occurs from the alpha or the bottom face and provides compounds where R³ is hydrogen.

Compounds of Formula (II) can be further elaborated as shown below:

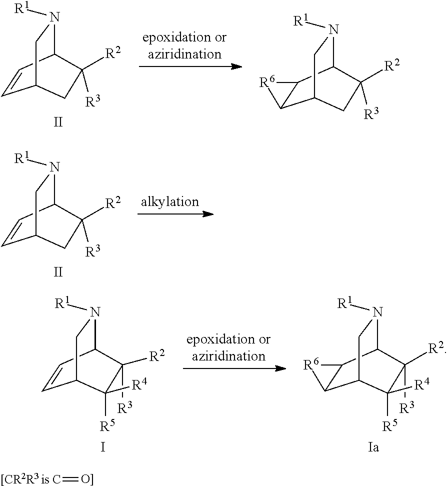

Methods of epoxidation and aziridination of double bounds are well known to the skilled artisan, and are performed, for example, with peracids such as percarboxylic acids, and for example, using p-toluene sulfonamide (TsNH₂) and an oxidant. Aziridines or protected aziridines, such as those provided herein, are also prepared by multi-step methods by first forming a geminal amino alcohol, protecting the amine, converting the alcohol to a leaving group (see supra), deprotecting the amine protection and cyclizing to form an aziridine which can be protected following methods well known to the skilled artisan.

More specifically, compound 6 is converted to compound 1 as illustrated schematically below:

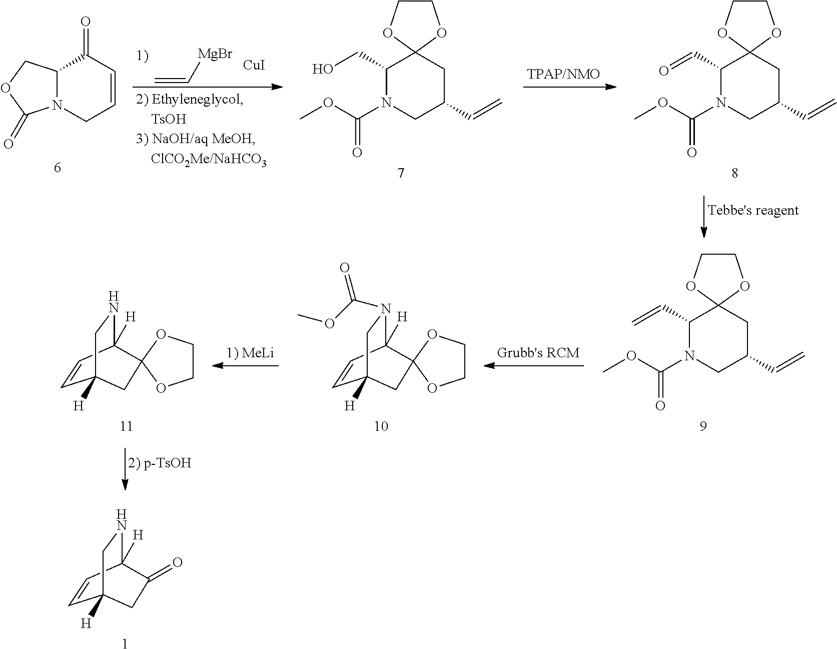

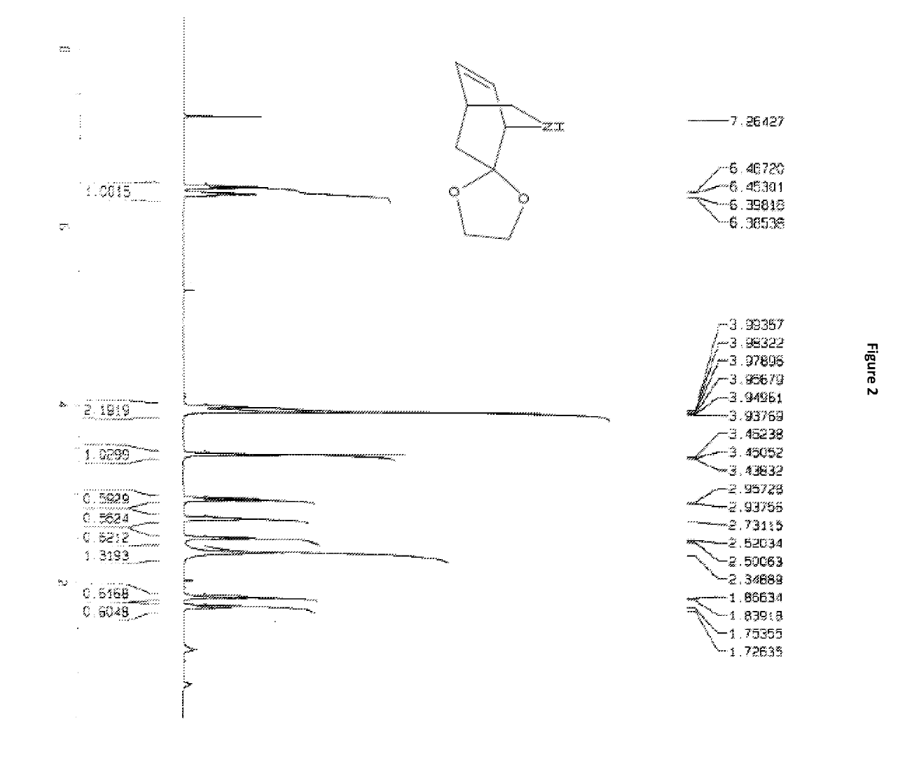

Conjugate addition of vinyl magnesium bromide, oxazolidine ring cleavage, and keto group protection provides compound 7. Compound 7 is oxidized using NMO and tetrapropylammonium perruthenate to provides compound 8. Olefination of 8 yields the 1,5 divinyl substrate piperidine (9). Grubbs cyclization of 2 yields optically active (0) which is the carbonyl group and N-protected derivative of the 1R,4R-2-azabicyclo[2,2,2]oct-5-ene-7-one ( ) mentioned above. The ¹H-NMR of compound 10 is provided in FIG. 1. Deprotection of the N-protecting groups of 10 provide compound 11, whose NMR is provided in FIG. 2. Deprotection of the carbonyl protection of 10 provides compound 1.

The isoquinuclidene compounds provided herein are also synthesized utilizing Diels Alder reactions as illustrated schematically below:

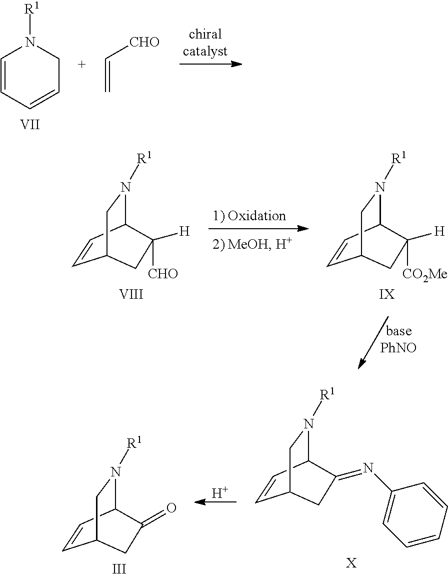

A Diels Alder reaction between compound VII, which is readily available, and acrolein, in presence of chiral catalysts, such as chiral Lewis acid catalysts provides compound VIII. In preferred embodiments, compound VIII is obtained in >99% ee. The aldehyde group in compound VIII is oxidized, following various well known methods, to a carboxylic acid and esterified to provide a carboxyl ester such as a methyl ester. Compound IX is decarboxylated by reacting with nitrosobenzene in presence of a base (such as, for example, hindered amide and silazide bases well known in the art) to provide Schiffs base X. Compound X is hydrolyzed to provide compound III. Compound III is conveniently elaborated to other compounds of this invention as shown above.

More specifically, a compound of this invention, compound 15, is synthesized as illustrated schematically below:

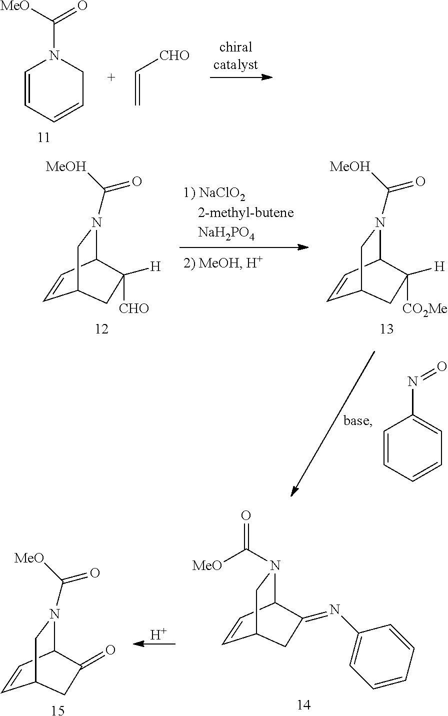

N-carbomethoxy-1,2-dihydropyridine is used as a starting material. Hypochlorite and 2-methyl-2-butene is used for oxidizing the —CHO group to a —CO₂H group.

Alternatively, compound III is synthesized using an acrylamide containing a chiral auxiliary as illustrated schematically below:

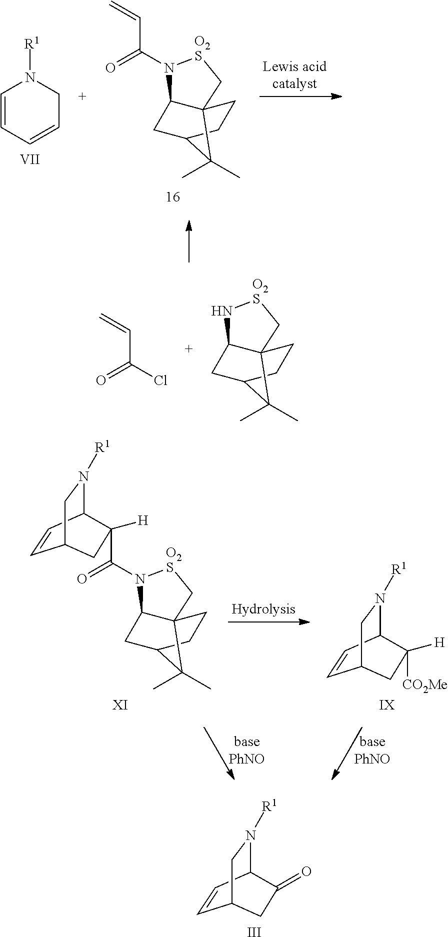

Various chiral auxiliaries useful for this purpose are well known in the art and the camphor based auxiliary is shown solely for illustration. In preferred embodiments, compound XI is obtained in >99% ee. Preferably, R¹ is a non-hydrogen substituent as defined herein.

More specifically, a compound of this invention, compound 15, is synthesized using N-carbomethoxy-1,2-dihydropyridine as a starting material and TiCl₄ as the Lewis acid catalyst as illustrated schematically below:

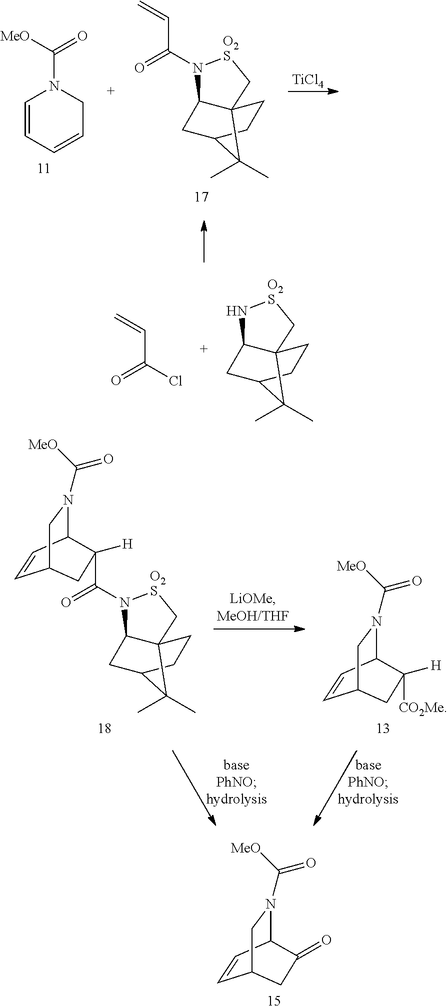

The reactions are carried out, preferably in an inert solvent that will be apparent to the skilled artisan upon reading this disclosure, for a period of time sufficient to provide a substantial amount of the product, which can be ascertained by using routine methods such as thin layer chromatography, ¹H-nuclear magnetic resonance (NMR) spectroscopy, and the likes. The products can be isolated and optionally purified using standard purification techniques, such as liquid chromatography, crystallization, precipitation, and distillation under reduced pressure, or the products may be used for a subsequent reaction without further purification.

### UTILITY

The compounds and processes provided herein have utility in synthesizing pharmaceutically active isoquinuclidene derivatives described for example in U.S. Pat. No. 6,211,360 and in synthesizing non-natural isoquinuclidene derivatives useful as 5-HT3 ligands (see, Iriepa et al., supra).

1. A compound of Formula (I) or (Ia): 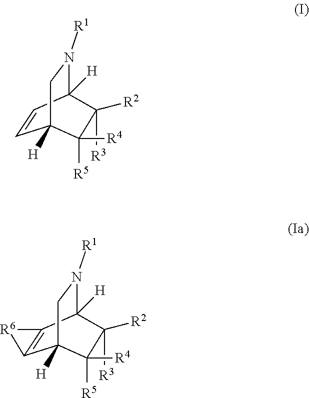
or a salt thereof wherein,
R¹ is selected from the group consisting of hydrogen, —CO₂R¹¹, —COR¹², —C(R¹³)₃, and an amine protecting group;
R¹¹ is selected from the group consisting of C₁-C₆ alkyl optionally substituted with 1-3 substituents selected from C₆-C₁₀ aryl, C₃-C₈ cycloalkyl, C₂-C₁₀ heteroaryl, C₃-C₈ heterocyclyl, halo, amino, —N₃, hydroxy, C₁-C₆ alkoxy, silyl, nitro, cyano, and CO₂H or an ester thereof, C₂-C₆ alkenyl, C₂-C₆ alkynyl, C₆-C₁₀ aryl, C₂-C₁₀ heteroaryl, C₃-C₈ cycloalkyl, and C₃-C₈ heterocyclyl,
R¹² and R¹³ independently are selected from the group consisting of hydrogen, C₁-C₆ alkyl optionally substituted with 1-3 substituents selected from C₆-C₁₀ aryl, C₃-C₈ cycloalkyl, C₂-C₁₀ heteroaryl, C₃-C₈ heterocyclyl, halo, amino, —N₃, hydroxy, C₁-C₆ alkoxy, silyl, nitro, cyano, and CO₂H or an ester thereof, C₂-C₆ alkenyl, C₂-C₆ alkynyl, C₆-C₁₀ aryl, C₂-C₁₀ heteroaryl, C₃-C₈ cycloalkyl, and C₃-C₈ heterocyclyl,
R² and R³ independently are hydrogen, hydroxy, C₁-C₆ alkyl, C₂-C₆ alkenyl, and C₂-C₆ alkynyl, —SR²¹ or —OR²², wherein the alkyl, alkenyl, or the alkynyl group is optionally substituted with 1-3 substituents selected from the group consisting of keto, halo, C₁-C₆ alkoxy, amino, hydroxy, cyano, nitro, —NHCOCH₃, —N₃, and —CO₂H or an ester thereof, provided that at least one of R² and R³, preferably R² is a non-hydrogen substituent, or
R² and R³ together with the carbon atom to which they are bonded to form a keto (C═O) group, a Schiff's base (═NR²⁴), a vinylidene moiety of formula ═CR²⁵R²⁶, or form a 5-6 membered cyclic ketal or thioketal, which cyclic ketal or thioketal of formula: 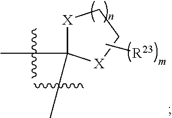
each R²¹ is independently selected from the group consisting of C₁-C₆ alkyl optionally substituted with 1-3 substituents selected from C₆-C₁₀ aryl, C₃-C₈ cycloalkyl, C₂-C₁₀ heteroaryl, C₃-C₈ heterocyclyl, halo, amino, —N₃, hydroxy, C₁-C₆ alkoxy, silyl, nitro, cyano, and CO₂H or an ester thereof, C₂-C₆ alkenyl, C₂-C₆ alkynyl, C₆-C₁₀ aryl, C₂-C₁₀ heteroaryl, C₃-C₈ cycloalkyl, and C₃-C₈ heterocyclyl;
each R²² is independently selected from the group consisting of C₁-C₆ alkyl optionally substituted with 1-3 substituents selected from the group consisting of C₆-C₁₀ aryl, C₃-C₈ cycloalkyl, C₂-C₁₀ heteroaryl, C₃-C₈ heterocyclyl, halo, amino, —N₃, hydroxy, C₁-C₆ alkoxy, silyl, nitro, cyano, and CO₂H or an ester thereof, C₂-C₆ alkenyl, and C₂-C₆ alkynyl;
X in both occurrences is either oxygen or sulfur;
m is 1, 2, 3, or 4;
n is 1 or 2;
R²³ is selected from the group consisting of C₁-C₆ alkyl and C₆-C₁₀ aryl;
R²⁴ is selected from the group consisting of C₆-C₁₀ aryl and C₂-C₁₀ heteroaryl;
R²⁵ is hydrogen, C₁-C₆ alkyl, C₂-C₆ alkenyl, and C₂-C₆ alkynyl, wherein the alkyl, alkenyl, or the alkynyl group is optionally substituted with 1-3 substituents selected from the group consisting of keto, C₁-C₆ alkoxy, amino, hydroxy, cyano, nitro, —NHCOCH₃, and —CO₂H or an ester thereof;
R²⁶ is hydrogen or C₁-C₆ alkyl;
R⁴ and R⁵ independently are selected from the group consisting of hydrogen, halo, C₁-C₆ alkyl optionally substituted with 1-3 substituents selected from the group consisting of C₆-C₁₀ aryl, C₃-C₈ cycloalkyl, C₂-C₁₀ heteroaryl, C₃-C₈ heterocyclyl, halo, amino, —N₃, hydroxy, C₁-C₆ alkoxy, silyl, nitro, cyano, vinyl, ethynyl, and CO₂H or an ester thereof,
R⁶ is selected from the group consisting of —O—, —NH—, and —NR⁶¹;
R⁶¹ is selected from the group consisting of hydrogen and an amine protecting group;
the amine protecting group is selected from the group consisting of —CO₂CMe₃, —CO₂Bn, —CO₂-allyl, -Fmoc (flurenyloxymethyl), —COCF₃, Bn (CH₂Ph), —CHPh₂, and —CPh₃;
wherein the cycloalkyl, heterocyclyl, aryl, or heteroaryl, is optionally substituted with 1-3 substituents selected from the group consisting of C₁-C₆ alkyl, C₂-C₆ alkenyl, C₂-C₆ alkynyl, C₆-C₁₀ aryl, cycloalkyl, C₂-C₁₀ heteroaryl, C₃-C₈ heterocyclyl, halo, amino, —N₃, hydroxy, C₁-C₆ alkoxy, silyl, nitro, cyano, and CO₂H or an ester thereof.

2. The compound of claim 1, of Formula (II): 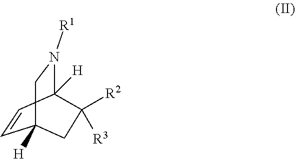
wherein R¹, R², and R³ are defined as in claim 1.

3. The compound of claim 2, wherein R¹ is hydrogen or CO₂R¹¹ and R^{II} is C₁-C₆ alkyl.

4. The compound of claim 2 wherein
R¹ is —CO₂R¹¹, —COR¹², —C(R¹³)₃, or another amine protecting group, wherein R¹¹ and R¹² defined as in claim 1 above,
R² is C₁-C₆ alkyl, C₂-C₆ alkenyl, or C₂-C₆ alkynyl, wherein the alkyl, alkenyl, or the alkynyl group is optionally substituted with 1-3 substituents selected from the group consisting of keto, halo, C₁-C₆ alkoxy, amino, hydroxy, cyano, nitro, —NHCOCH₃, —N₃, and —CO₂H or an ester thereof, and
Z³ is hydroxy or hydrogen.

5. The compound claim 1 of Formula (IIA): 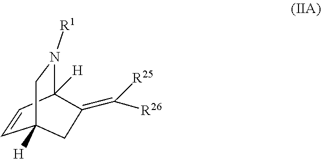
wherein R¹, R²⁵, and R²⁶ are defined as in Formula (I) above.

6. A compound of formula: 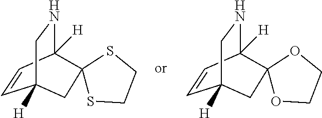 [SMILES: C1=C[C@@H]2CNC1C1(C2)SCCS1 ; C1=C[C@@H]2CNC1C1(C2)OCCO1]
or a salt thereof.

7. The compound of claim 5, which is an R,R enantiomer.

8. An isolated R,R enantiomer of the compound of claim 7, which is in substantial enantiomeric excess (ee).

9. A process for preparing a compound of Formula (II) 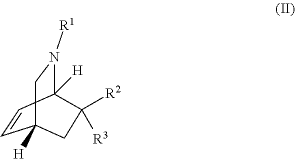
or a salt thereof, wherein
R¹ is selected from the group consisting of hydrogen, —CO₂R¹¹, —COR¹², —C(R¹³)₃ and an amine protecting group;
R¹¹ is selected from the group consisting of C₁-C₆ alkyl optionally substituted with 1-3 substituents selected from the group consisting of C₆-C₁₀ aryl, C₃-C₈ cycloalkyl, C₂-C₁₀ heteroaryl, C₃-C₈ heterocyclyl, halo, —N₃, hydroxy, C₁-C₆ alkoxy, silyl, nitro, cyano, and CO₂H or an ester thereof, C₂-C₆ alkenyl, C₂-C₆ alkynyl, C₆-C₁₀ aryl, C₂-C₁₀ heteroaryl, C₃-C₈ cycloalkyl, and C₃-C₈ heterocyclyl,
R¹² and R¹³ independently are selected from the group consisting of hydrogen, C₁-C₆ alkyl optionally substituted with 1-3 substituents selected from the group consisting of C₆-C₁₀ aryl, C₃-C₈ cycloalkyl, C₂-C₁₀ heteroaryl, C₃-C₈ heterocyclyl, halo, —N₃, hydroxy, C₁-C₆ alkoxy, silyl, nitro, cyano, and CO₂H or an ester thereof, C₂-C₆ alkenyl, C₂-C₆ alkynyl, C₆-C₁₀ aryl, C₂-C₁₀ heteroaryl, C₃-C₈ cycloalkyl, and C₃-C₈ heterocyclyl,
the amine protecting group is selected from the group consisting of —CO₂CMe₃, —CO₂Bn, —CO₂-allyl, -Fmoc (flurenyloxymethyl), —COCF₃, Bn (CH₂Ph), —CHPh₂, and —CPh₃;
R² and R³ independently are selected from the group consisting of —S—R²¹ and —OR²², or R² and R³ together with the carbon atom to which they are bound form a keto (C═O) group or form a 5-6 membered cyclic ketal or thioketal of formula: 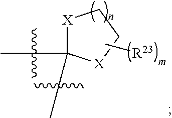
each R²¹ is independently selected from the group consisting of C₁-C₆ alkyl optionally substituted with 1-3 substituents selected from the group consisting of C₆-C₁₀ aryl, C₃-C₈ cycloalkyl, C₂-C₁₀ heteroaryl, C₃-C₈ heterocyclyl, halo, —N₃, hydroxy, amino, C₁-C₆ alkoxy, silyl, nitro, cyano, and CO₂H or an ester thereof, C₂-C₆ alkenyl, C₂-C₆ alkynyl, C₆-Cₜ₀ aryl, C₂-C₁₀ heteroaryl, C₃-C₈ cycloalkyl, and C₃-C₈ heterocyclyl;
each R²² is independently selected from the group consisting of C₁-C₆ alkyl optionally substituted with 1-3 substituents selected from the group consisting of C₆-C₁₀ aryl, C₃-C₈ cycloalkyl, C₂-C₁₀ heteroaryl, C₃-C₈ heterocyclyl, halo, amino, —N₃, hydroxy, C₁-C₆ alkoxy, silyl, nitro, cyano, and CO₂H or an ester thereof, C₂-C₆ alkenyl, and C₂-C₆ alkynyl;
X is in both occurrences are O or S;
m is 1, 2, 3, or 4;
n is 1 or 2;
R²³ is selected from the group consisting of C₁-C₆ alkyl and C₆-C₁₀ aryl;
wherein the cycloalkyl, heterocyclyl, aryl, or heteroaryl, is optionally substituted with 1-3 substituents selected from the group consisting of C₁-C₆ alkyl, C₂-C₆ alkenyl, C₂-C₆ alkynyl, C₆-C₁₀ aryl, C₃-C₈ cycloalkyl, C₂-C₁₀ heteroaryl, C₃-C₈ heterocyclyl, halo, amino, —N₃, hydroxy, C₁-C₆ alkoxy, silyl, nitro, cyano, and CO₂H or an ester thereof;
which process comprises contacting a compound of Formula (IV): 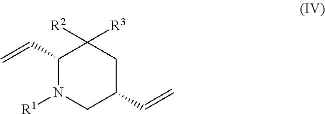
or a salt thereof wherein, R¹, R², and R³ are defined as in formula (III) above,
with less than 1 molar equivalent of an olefin metathesis reagent under conditions to provide a compound of Formula (II) or a salt thereof.
